(19) United States
(12) Patent Application Publication
(10) Pub. No.: US 2026/0236095 A1
(43) Pub. Date: Aug. 13, 2026

(54) AUTOMATIC GAZE ESTIMATION

(71) Applicant: KING'S COLLEGE LONDON, London, Greater London (GB)

(72) Inventors: Kun QIAN, London (GB); Tomoki ARICHI, London (GB); Joseph V. HAJNAL, London (GB)

(21) Appl. No.: 19/470,722

(22) PCT Filed: Mar. 27, 2024
(86) PCT No.: PCT/GB2024/050840

§ 371 Date: Sep. 29, 2025

(30) Foreign Application Priority Data
Mar. 29, 2023 (GB) 2304577.6

(51) Int. Cl. G06F 3/01; G06T 7/73

(52) U.S. Cl. CPC G06F 3/013; G06F 3/012; G06T 7/73; G06T 2207/20081; G06T 2207/30201

(57) ABSTRACT

Aspects and embodiments provide an apparatus and method to auto-calibrate an eye tracking system for a user. A method in accordance with aspects and embodiments may comprise: providing a model linking eye data with a gaze position on a display, wherein the model is configured to link user eye feature location and an indication of user head location to a gaze location; acquiring user eye data and transforming the acquired user eye data into local eye image space to obtain an indication of user eye feature location and indication of user head location in centralised eye image space; providing an icon at a known position on the display, the icon being associated with a surrounding collider area and transforming the known position to accord with local eye image space; estimating, based upon the model and the transformed acquired user eye data and the transformed known position, whether a user gaze position falls within the collider area; and if the estimated user gaze position is determined to fall within the collider area, associating the transformed acquired user eye data with the transformed known position and adapting the model linking eye data with gaze position based upon the transformed known position and associated transformed acquired eye data. Use of a local eye space based gaze estimation model can help to support a reduction in large gaze errors caused by dataset variations, which may be of particular use when initialising a gaze tracking system for use by a particular user, thereby allowing a gaze tracking system to support substantially instant effective user interaction without a need for an explicit calibration phase.

### FIELD OF THE INVENTION

Aspects and embodiments relate to a method of auto-calibrating an eye gaze tracking system for a user, and an apparatus and computer program product configured to perform such a method.

### BACKGROUND

Human-computer interaction (HCI) increasingly forms part of everyday life. Making such interaction people-oriented is a key design principle and seeks to ensure that a workload for a human is minimised to result in efficient operation of the computer-human pairing.

People-orientation of HCI may be evident in relation to interface design. Instant control of a user interface is perceived as important to users of a computer, especially young children, since, if appropriately implemented, it can make interaction with a computer easy, fast and intuitive.

Various mechanisms for a human to interact with a computer are possible including, for example, use of a keyboard, mouse, touch screen, use of voice and similar. One such mechanism comprises gaze tracking, in which a fixation point of a user as they look at, for example, a screen or scene is computed from monitored eye features. Monitorable eye features include, for example, position of user pupil, corneal reflection and similar. Gaze control is increasingly being used to enrich HCI.

Gaze control interfaces form the core of gaze interaction as a mechanism for HCI and operate to convert an estimated gaze location on a screen into an output which can be used to control and/or communicate with, for example, a graphic user interface (GUI) of a computer system.

Systems including gaze control as an interaction mechanism may not be instantly or effortlessly usable by system users. There are multiple suppliers of camera systems designed for providing high quality eye feature tracking. Despite such camera systems, gaze tracking results remain unsatisfactory, often having high and/or unpredictable errors, for example, in relation to a difference between an estimated on-screen gaze position and a “true” gaze position. The unpredictable error can be caused by various factors. One major factor in gaze estimation error is head motion.

Even small changes in head position can completely disrupt many of the gaze estimation systems. Another major factor in gaze estimation error results from calibration processes. Gaze estimation typically requires a personalised model to compute a fixation point on the screen from user eye feature data. To create such a personalised model, a calibration procedure is normally needed which requires a user to stare at a plurality of calibration points (typically 9 discrete points). The data obtained as part of the calibration process allows for collection of sufficient data to “solve” the personalised calibration model. A gaze estimation system calibration procedure is typically time consuming and requires a period of intense user cooperation. The calibration procedure is therefore sometimes challenging for users, especially young users.

Gaze estimation systems may be configured to implement open loop calibration. Accordingly, after an initial calibration process is completed, a system is assumed to remain in calibration for the rest of a session, and there is no further check of whether the estimated gaze point corresponds to a true gaze location. Since the eye tracker's performance and resulting gaze estimation processes degrade with user head movement, the gaze estimation may degrade in a session to the extent that the calibration step has to be performed again. A need to repeat the calibration process has a significant impact upon user experience.

It is possible to ease the burden of initial calibration and ongoing maintenance of performance of a gaze tracker by implementing one or more implicit calibration method.

Implicit calibration procedures are configured such that they pair a predicted gaze location, which is obtained via a prior or initial gaze estimation model, and corresponding eye feature data obtained from an eye tracker. The captured eye position data is used in a way similar to the explicit calibration steps set out above to enhance the initial gaze estimation model.

A key challenge in an implicit model remains how to infer gaze or detect user attention without having obtained an initial good quality personalised gaze estimation model.

Implicit calibration methods may rely on a user implementing a specific hardware input, for example, mouse click, key press, fully calibrated device or similar. Alternatively, or additionally, implicit calibration methods may rely on a system being configured to implement extra user tasks, for example, target pursuit, salience map or video watching, or additional hand eye coordination tasks. Whether hardware input or additional user tasks, such implicit calibration methods are such that they use information acquired during those processes to build an initial model and infer gaze.

Personalized models are of limited use in relation to new users. Each user is likely to have different eye features, for example, eye size, shape and similar. Furthermore, personalised models do not account well for different user head poses.

Whilst it is possible to reduce the influence of eye feature variation on applicability of a model using techniques such as normalization and deep learning, such approaches may rely on knowledge of a full face coordinate for data alignment, which may be of limited applicability in scenarios where full face information is not available.

It is desired to provide a mechanism which may address one or more issue associated with gaze estimation.

### SUMMARY

According to some, but not necessarily all, aspects of the invention there is provided a method of auto-calibrating an eye gaze tracking system for a user, the method comprising: providing a model linking eye data with a gaze position on a display, wherein the model is configured to link user eye feature location and an indication of user head location to a gaze location; acquiring user eye data and transforming the acquired user eye data into local eye image space to obtain an indication of user eye feature location and indication of user head location in centralised eye image space;

providing an icon at a known position on the display, the icon being associated with a surrounding collider area and transforming the known position to accord with local eye image space; estimating, based upon the model and the transformed acquired user eye data and the transformed known position, whether a user gaze position falls within the collider area; and if the estimated user gaze position is determined to fall within the collider area, associating the transformed acquired user eye data with the transformed known position and adapting the model linking eye data with gaze position based upon the transformed known position and associated transformed acquired eye data. Use of a local eye space based gaze estimation model can help to support a reduction in large gaze errors caused by dataset variations, which may be of particular use when initialising a gaze tracking system for use by a particular user, thereby allowing a gaze tracking system to support substantially instant effective user interaction without a need for an explicit calibration phase.

In some embodiments, the user eye feature location comprises user eye pupil location.

In some embodiments, the indication of user head location comprises an indication of a user facial anchor. Such facial anchor information may comprise an indication of a rigid or substantially fixed facial reference point location such as: eye corner or corners, nose, face outline, or combination of such features.

In some embodiments, the model comprises a model which maps local eye image space to a gaze position on a display which has been transformed into an equivalent transformed gaze space.

In some embodiments, local eye image space comprises a coordinate system in which an eye tracking origin coordinate used when acquiring user data from an eye image is transformed and scaled to map to an image centre.

In some embodiments, transformed known positions comprise display locations which have been transformed to account for origin and scale transformations applied to data from an eye image.

In some embodiments, the icon comprises a visual target and an associated virtual collider area.

In some embodiments, the virtual collider area has a diameter larger than the diameter of the visual target. In some embodiments, the virtual collider area of an initial visual target shown to a user may have a diameter at least twice the diameter of the initial visual target.

In some embodiments, the collider area is centred upon the known position.

In some embodiments, the icon comprises an adaptive icon at a known position on the display, the adaptive icon being configured to adapt when the model and acquired eye data estimate that the user gaze position falls within the collider area.

In some embodiments, the method comprises: adapting the icon whilst the estimated user gaze position is determined to fall within the collider.

In some embodiments, the method comprises performing the steps of:

acquiring further user eye data and transforming the further acquired user eye data into local eye image space to obtain an indication of user eye feature location and indication of user head location in centralised eye image space; providing an icon at a further known position on the display, the icon being associated with a surrounding collider area and transforming the further known position to accord with local eye image space; estimating, based upon the model and transformed further acquired user eye data, and the transformed further known position whether a user gaze position falls within the collider area; and if the estimated user gaze position is determined to fall within the collider area, associating the further transformed acquired further user eye data with the transformed further known position and further adapting the model linking eye data with gaze position based upon the transformed further known position and associated transformed further acquired eye data.

In some embodiments, the method comprises iterating the model linking eye data with a gaze position on a display to auto-calibrate for a user by repeating the steps set out above.

In some embodiments, the method comprises changing the size of the collider area as part of a series of iterative steps to refine the auto-calibration of the eye gaze tracking system for the user.

In some embodiments, changing the size of the collider area comprises: reducing the relative size of the collider area compared to the icon.

In some embodiments, the known position comprises a position substantially in the centre of the display

In some embodiments, the further known position comprises a position at the periphery of the display.

According to some, but not necessarily all, aspects there is provided a computer program product operable, when executed on a computer, to perform the method set out above.

According to some, but not necessarily all, aspects there is provided an auto-calibrated gaze estimation apparatus comprising at least one processor; and at least one memory storing instructions that, when executed by the at least one processor, cause the gaze estimation apparatus to: provide a model linking eye data with a gaze position on a display, wherein the model is configured to link user eye feature location and an indication of user head location to a gaze location; acquire user eye data and transforming the acquired user eye data into local eye image space to obtain an indication of user eye feature location and indication of user head location in centralised eye image space; provide an icon at a known position on the display, the icon being associated with a surrounding collider area and transforming the known position to accord with local eye image space; estimate, based upon the model and the transformed acquired user eye data and the transformed known position, whether a user gaze position falls within the collider area; and if the estimated user gaze position is determined to fall within the collider area, associate the transformed acquired user eye data with the transformed known position and adapt the model linking eye data with gaze position based upon the transformed known position and associated transformed acquired eye data.

In some embodiments, the user eye feature location comprises user eye pupil location.

In some embodiments, the indication of user head location comprises an indication of a user facial anchor. Such facial anchor information may comprise an indication of a rigid or substantially fixed facial reference point location such as: eye corner or corners, nose, face outline, or combination of such features.

In some embodiments, the model comprises a model which maps local eye image space to a gaze position on a display which has been transformed into an equivalent transformed gaze space.

In some embodiments, local eye image space comprises a coordinate system in which an eye tracking origin coordinate used when acquiring user data from an eye image is transformed and scaled to map to an image centre.

In some embodiments, transformed known positions comprise display locations which have been transformed to account for origin and scale transformations applied to data from an eye image.

In some embodiments, the icon comprises a visual target and an associated virtual collider area.

In some embodiments, the virtual collider area has a diameter greater than the diameter of the visual target.

In some embodiments, the collider area is centred upon the known position.

In some embodiments, the icon comprises an adaptive icon at a known position on the display, the adaptive icon being configured to adapt when the model and acquired eye data estimate that the user gaze position falls within the collider area.

In some embodiments, the apparatus is configured to: adapt the icon whilst the estimated user gaze position is determined to fall within the collider.

In some embodiments, the apparatus is configured to perform the steps of: acquiring further user eye data and transforming the further acquired user eye data into local eye image space to obtain an indication of user eye feature location and indication of user head location in centralised eye image space; providing an icon at a further known position on the display, the icon being associated with a surrounding collider area and transforming the further known position to accord with local eye image space; estimating, based upon the model and transformed further acquired user eye data, and the transformed further known position whether a user gaze position falls within the collider area; and if the estimated user gaze position is determined to fall within the collider area, associating the further transformed acquired further user eye data with the transformed further known position and further adapting the model linking eye data with gaze position based upon the transformed further known position and associated transformed further acquired eye data.

In some embodiments, the apparatus is configured to iterate the model linking eye data with a gaze position on a display to auto-calibrate for a user by repeating the steps set out above.

In some embodiments, the apparatus is configured to change the size of the collider area as part of a series of iterative steps to refine the auto-calibration of the eye gaze tracking system for the user.

In some embodiments, changing the size of the collider area comprises: reducing the relative size of the collider area compared to the icon.

In some embodiments, the known position comprises a position substantially in the centre of the display.

In some embodiments, the further known position comprises a position at the periphery of the display.

Further particular and preferred aspects are set out in the accompanying independent and dependent claims. Features of the dependent claims may be combined with features of the independent claims as appropriate, and in combinations other than those explicitly set out in the claims. Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which: FIG. 1 illustrates schematically some main stages of a general adaptive instant gaze estimation approach in accordance with some arrangements;

FIG. 2A to FIG. 2E provides a visual comparison of local coordinate and global coordinate based eye feature representation.

FIG. 3A illustrates a screen display region in accordance with an arrangement;

FIG. 3B illustrates a gaze interaction target in accordance with an arrangement;

FIG. 4 is a graphical illustration of a performance comparison between various possible gaze control methods; and

FIG. 5 illustrates graphically a confidence interval of a gaze modelling process in accordance with arrangements.

### DESCRIPTION OF THE EMBODIMENTS

When providing a system in which human computer interaction (HCI) is supported, ensuring interaction is instant and effortless is an important characteristic which enhances user experience. Instant control user interfaces can help reduce user frustration, increase user engagement, and encourage exploration of the system by a user.

In most gaze control applications, instant interaction cannot be achieved because the applications require a user to cooperate to complete a laborious and sometimes error-prone explicit calibration procedure. That calibration procedure allows an application to build an initial gaze estimation model. Often such gaze estimation models become irrelevant and no longer applicable if a user moves their head.

Explicit calibration methods may be supported or replaced by implicit calibration techniques. Such implicit calibration techniques embed a calibration procedure into normal user activities such as, for example, video watching, or key pressing. A key feature of implicit calibration is that gaze estimation error is dynamic and normally large in initial phases. The large error is a result of a lack of initial personalised model. Not having an initial personalised model hinders instant gaze interaction. To improve an initial generic model and provide a personalised model, implicit calibration techniques require specific hardware input such as, for example, mouse click, key press, fully calibrated device and similar or extra user tasks, for example, target pursuit, salience map or video watching, and hand-eye coordination events. Such specific hardware input or extra user tasks take time, increase user learning burden, and may not be compatible with the general gaze control interface.

Arrangements described seek to reduce the influence of a need for a personalised model in initial stages of commissioning or establishing a gaze estimation system. In support of such a reduction, arrangements described may implement a novel local-space-representation based gaze model. Such a model can reduce the influence of eye feature and initial head pose variation under small head motion. By contrast, a global coordinate representation is based on a fixed global reference which is sensitive to eye feature and head pose variation, so errors in gaze estimation in a global coordinate system can often be introduced as part of the initialisation phase of a system.

Arrangements described may provide a local space based gaze estimation model which is less influenced by eye features (such as, for example, eye shape and size) and initial head pose variation under small head motion. Furthermore, arrangements described may provide small gaze error in the beginning phase and support instant user interaction.

Based on an appropriate gaze estimation model, arrangements may provide an interaction driven adaptive gaze control interface. Whilst the interaction driven adaptive gaze control interface has been demonstrated to have efficacy in conjunction with the gaze estimation model described, it will be appreciated that it may also be used with other, more complex or alternative initial models.

Arrangements may provide an integration strategy configurable to integrate with general interface design.

Example arrangements are described in relation to a challenging Magnetic Resonance Imaging (MRI) scanner environment. By way of background, whilst the general applicability of the approaches and arrangements described has been realised, a gaze control based MRI compatible Virtual Reality (VR) system was the catalyst for creation of various described arrangements.

The gaze control based MRI-compatible VR system is primarily used for reducing patient anxiety during MRI scanning. That system underlined the importance of providing a mechanism for instant interaction in an HCI system. The instant interaction feature was found to be particularly useful in supporting an HCI system which can be utilised by young children. The system used to demonstrate arrangements described does not rely upon provision of external hardware input and/or completion of extra user cooperation tasks. It will thus be appreciated that whilst described in relation to an MRI application, the gaze estimation approaches detailed below have the potential to be applied to a general-purpose gaze interaction system.

An advantage of approaches in line with described arrangements is that eye feature mapping is more closely related to gaze direction change as a result of use of local representation techniques. Based on the local representation techniques, arrangements may provide an interaction driven gaze control interface. Some arrangements are configured such that they support instant and continuous gaze interaction between a user and an HCI system. A gaze control interface application is described below in the context of a Virtual Reality (VR) based patient experience improvement system for Magnetic Resonance Imaging (MRI) exams. The results of a demonstration of approaches in that context indicate that an instant gaze interface in accordance with arrangements may support instant user interaction with a system via the gaze mechanism, thus encouraging user engagement with the system and reducing user frustration by comparison to a system which implements traditional calibration-style tasks.

Gaze Tracking Methods Gaze estimation methods fall into several categories: geometric based, 2D feature mapping based and appearance based.

Geometric based methods combine eye-in-head rotations and head-in-world coordinates to derive a gaze vector and its intersection with a planar display. Geometric based methods normally require complex lighting and a specific camera setup to infer eye geometry and are unlikely to be suitable for use in dynamic and complex environments.

2D feature mapping based methods estimate gaze by building a mapping function between eye movement features and gaze. Eye movement features include: pupil/iris-corner technique (PCT/ICT)-based methods, pupil/iris-corneal reflection technique (PCRT/ICRT)-based methods, cross-ratio (CR)-based methods, and homography normalization (HN)-based methods. Construction of a mapping function between eye movement features of a user and gaze according to 2D feature mapping techniques requires explicit personal calibration and the static mapping is vulnerable to even small head motion.

Appearance based methods enable eye appearance images to be directly mapped to desired coordinates for gaze. Although the appearance based approaches are, in principle, suited to complex and dynamic scenarios, they require sufficient data and long training hours to create a gaze model. That gaze model is unlikely to have a level of accuracy which supports reliable gaze interaction and control, instead operating as a mechanism for gaze to indicate attention detection. Appearance based methods rely on use of training data which cannot cover all eye appearance variations resulting from different head poses, camera distances, lighting conditions and similar. Whilst a range of techniques for reducing the impact of eye feature variation are possible, including, for example, the use of normalization techniques and deep learning-based approaches, they rely on full facial information (face coordinate system). In many scenarios, full facial features are not available, for example, in relation to wearable eye tracking devices which use near-eye eye tracking images, or in relation to image capture of a partially occluded face.

A large obstacle to instant gaze interaction relates to creation of a good quality personalized gaze model. To create such a model, a user of a gaze estimation system may be required to complete a laborious and often error prone calibration procedure.

In recognition of the obstacle and to overcome the model creation challenge, implicit calibration methods may be used.

### Implicit Calibration

Some implementations of implicit calibration methods for gaze estimation rely on manual input in combination with eye imaging. That manual input may, for example, comprise use of a mouse click to assist in inferring gaze location. However, such methods rely on external hardware to infer gaze which can limit the extent to which they can be used in all applications.

Requiring users to perform click tasks allows for collection of sufficient eye position images as samples to map to positions known as a result of the click tasks to build an appropriate gaze estimation model which can then be used for ongoing user interaction with a system. The method proposed in (6) achieved a gaze error above 4 degree after 1000 clicks. In (7), users have to play a click game for at least 20 s to achieve an initial gaze model which can predict 2181 out of 2886 clicks (about 4 degree error). In (8), an error of around 2 degree was achieved after 1500 mouse and keyboard clicks.

A more general implicit calibration method may be implemented based on a visual saliency map. Such an approach was proposed in (9) which provides a function to estimate the probability of gaze location on the screen. The dataset preparation of (9) is based on extracting eye image and saliency map pairings simultaneously whilst subjects watch a video clip. The training 10 clips of videos (7500 frames) in (9) can achieve an error of 3 degree. That degree of error is enough to detect regions of attention, but not enough to allow fine control of a system through gaze interaction with the system.

Although methods have been proposed to improve the performance of saliency map approaches, they typically require users to passively watch a sequence of images for a long time. For example, the methods proposed in (10) and (11) require a user to watch 10 images (4 seconds each) and one image (30 seconds) respectively and the resulting gaze estimation accuracy is larger than 1.0 degree error with a user head in a fixed position.

Methods based on a saliency map require user cooperation and the resulting inferred gaze may be unreliable if the user does not focus on the video or image stimuli.

Fully calibrated device-based methods (12) require a knowledge of camera, light source and monitor positions (13). Such a strict adherence to a rigid physical set up is not practical for use in many environments. Methods based on a fully calibrated device use positioning information, for example, known Euclidean relationships as a result of a calibrated setup, to estimate an optical axis. By showing a user at least one calibration point, an offset to a visual stimulus can be estimated. Although one point calibration achieves an improvement from a user experience perspective compared to a multiple point calibration process, the system setup requires laborious geometric calibration. Furthermore, any slight change of components of the system, for example, a camera lens, or light source position, is likely to influence the accuracy of the gaze system.

To reduce dependency on a fully calibrated device, cross-ratio based methods can be implemented ((14) to (18)). Such methods rely on projective invariants of corneal reflection to build a gaze estimation model. To maintain a reflection on the corneal surface needs a user's cooperation. Maintaining the position of the reflection highly constrains behaviour of a user and is not practical in many scenarios.

Pursuit calibration as used in ((19) to (21)) is a type of implicit calibration based upon a smooth pursuit eye movement that a user naturally performs when following a moving object with our eyes. In (20) the correlation of eye movement trajectory and target movement trajectory is used for detecting user attention and gaze sample collection. A 10 s target pursuit can achieve an error of around 1 degree. In (21) pursuit calibration is used in relation to difficult-to-calibrate participants, for example, users having Attention Deficit Hyperactivity Disorder (ADHD). The techniques set out in (21) require use of a 30-60 second target pursuit procedure and achieved an accuracy of around 2 degree.

Although pursuit calibration can improve calibration flexibility and rigidity, it still requires users to cooperate and perform extra user tasks.

Hand-eye coordination during interaction has been extensively studied in neuroscience (22). Humans regularly use their eyes to guide and coordinate their hands when interacting. In (23) implicit calibration is performed based upon hand interactions in VR. Such hand interactions include gestures such as: release, reach and manipulation. The approach described in (23) showed that 1 minute of user operation, during which over 300 gaze samples were collected, can achieve gaze estimation having about 1 degree error. (23) sets out that the size of the object with which a user interacts has an impact upon the success of the calibration method. A target size having 1 degree of the field of view is recommended. However, the main limitation of the study was that it required standard calibration to initiate the process and only considered a small number of common interactions with a single object.

It will be appreciated that whilst various methods have the potential to support instant and continuous gaze interaction, their compatibility with a general gaze interaction interface may be limited. In particular, some methods described above are subject to: restrictions on specific hardware combinations such as a mouse click or provision of a fully calibrated device; extra procedures or user tasks such as: target pursuit, video watching, object manipulation. Furthermore, gaze errors resulting from the various methods described above are progressive, and normally large in initial phases. As a result, such methods require an interaction interface to tolerate a dynamic error. Accounting for dynamic errors is not considered in most application designs.

Gaze Interaction Interfaces

Gaze interaction interfaces allow users to control a graphic user interface (GUI) using gaze. Gaze interaction interfaces can be adapted in various ways. Some gaze interaction interfaces can focus on improving gaze selection robustness and accuracy. The core idea of such interfaces is that of dynamically adapting GUI design and interaction techniques to match quality of gaze tracking. Error-aware gaze interaction interfaces may operate such that rather than updating a gaze model using an observed gaze sample (as in the implicit calibration approaches described above), they embrace gaze error and apply predictive models to correct gaze on the fly. The interface may be configured to dynamically adjust a UI layout and UI element sizes according to gaze error. However, such methods cannot deal with gaze tracking performance degradation issues. In order to dynamically change UI layout and button/element size to adapt to a current gaze error typically results in a user learning burden.

Some gaze interaction interfaces focus on GUI design and user experience. Some such interfaces use “multiple confirm” as a way to avoid inadvertent user selection of an option. The core idea is that the fixation of a user gaze on an interactive target results in the activation of a new UI element. That activated UI element may comprise element(s) having various shapes and layers; result in magnification of a sub-region; and/or zoom around the gaze point of fixation, which can be useful to a user for small object selection. However, “pop-up” or “activated” interfaces can be associated with a loss of context to a user and may also have a high learning threshold for a user.

“Multiple confirm” interfaces assume that input gaze estimation is accurate and often require use of the laborious explicit calibration procedures set out above.

Whilst gaze interaction interfaces may take into account adapting GUI design to take into account gaze estimation error, such a dynamic layout concept cannot be easily extended to apply to existing applications.

Gaze interaction interfaces typically rely on use of explicit calibration methods, rather than the implicit calibration methods set out above.

Approaches in accordance with arrangements described herein recognise that it is possible to develop a adaptive gaze control interface which is less affected by personalized eye feature variation, including, for example, eye shape, eye size variation and similar; can tolerate small degrees of head motion and also supports substantially “instant” gaze interaction with an interface by a user.

Approaches described may allow for creation of a gaze control interface in which it is possible to adapt the interface, as gaze estimation accuracy varies, so that applications can be provided in which it is not necessary to redesign an entire GUI to accommodate gaze estimation accuracy variation. Approaches in accordance with some arrangements may provide an interface which supports adjustment of a current GUI to adapt to adaptive gaze estimation errors.

### Instant Gaze Pipeline

A system which utilises various components is now described in detail. It will be appreciated that the elements of the various components described may be used in other contexts, without elements of other described components. For example, the described gaze estimation model may be used without the implicit calibration method described. In other words, the adaptive gaze estimation techniques described may be used without the initial model and data processing described, which in turn can be used without the local based feature construction and so on.

The system in which example implementations of arrangements have been tested comprise an interactive display system based on an MR compatible Virtual Reality (VR) system developed for patient distraction as described in detail in (34). The system comprises various components including: a customized 3D printed coil mount VR headset and a MR compatible projector (SV-8000 MR-Mini, Avotec Inc, Florida). A projector operating in such a system is configured to project VR content, for example, from a computer outside an MRI scanner room via an optical fibre data link, onto a screen of the headset. The headset of the example system comprises: a screen, a viewing mirror and two cameras configured to collect images of the eyes in support of eye tracking operations. A user of the system is positioned to see the visual content projected onto the screen via the viewing mirror. The two cameras (12M-I, MRC Systems, Heidelberg) each have an infrared illuminator diode. The system is configured to use the cameras to capture one or more images of an eye region of a user. That image capture may occur in real time. The cameras may be configured to provide a high resolution eye image (640×480) to the system for use in gaze estimation processes. More details about the hardware setup can be found in Qian et al. (34).

FIG. 1. illustrates schematically some main stages of an adaptive instant gaze estimation approach in accordance with some arrangements. An initial set of eye images 100 is collected. The eye images may comprise data from which gaze can be estimated. Each eye image can be analysed to extract coordinates of eye features and the eye feature information is associated with gaze target information. The eye image samples and associated gaze target information pairs are collected from a total of X subjects. In accordance with some arrangements, steps can be taken to process the eye images and extract or identify key eye features and associate them with corresponding target coordinates. That initial set of basic information pairings can, in accordance with some arrangements, be transformed or converted into a different space or coordinate system. In other words, the eye feature data may be transformed 101 into a selected coordinate system selected to best map eye features to likely gaze, taking into account head motion, and the target data may be translated or transformed 101 to take account of the applied eye data coordinate transformation. A weighting may be applied to each data pairing (transformed eye data, transformed target data) and a dataset 140 upon which a gaze model can be solved can be created.

Based on the processed dataset 140, a gaze model 150 can be solved. That gaze model can be used within a gaze control interface 155 to allow a user to interact with a system. In accordance with the implementation shown schematically in FIG. 1, each time a user performs a gaze interaction 160 within a system, a new sample 170 can be collected, converted into a local space representation 180 and added to the dataset 140. The gaze model 150 can then be iteratively or sequentially updated based on the “new” dataset which includes the new sample 170.

### Adaptive Gaze Estimation

Having described the main stages of the adaptive gaze estimation approach in relation to FIG. 1 a more detailed description of some possible implementations is provided. Some implementations of approaches in accordance with described arrangements provide a deep learning based (using Yolo framework as in (32)) eye tracker which is configured to analyse captured eye images and extract, for example, eye feature information. Such eye feature information may comprise: eye pupil centre (px, py); and facial anchor (m,n) information.

The eye tracking results are used, according to some arrangements, to construct an eye feature vector e as explained further below.

According to some arrangements, a simple and low computational cost step of performing polynomial regression occurs to map eye feature vector e to a 2D gaze point on the screen g∈R^{1×2}.

According to one possible arrangement, the gaze estimation model f for each eye is defined as:

$$g = f ( e ) = E ( x , y ) + H ( m , n ) \qquad [ 1 ]$$

Where f(e) is made up of two terms:
E(x,y) (a relative eye feature term) and
H(m,n) (a head motion influence term).

The relative eye feature term reflects a gaze direction change. The head motion influence term according to adopted approaches uses facial anchor positions as a reflection of head motion. Some approaches, using head motion and facial anchor positions, assume that the head movement is relatively small. Such facial anchor positions may comprise various positions which can be identified and which remain substantially constant relative to, for example, pupil location, as gaze changes. By way of example, possible facial anchor features include: eye corner(s), edge of face, nose position and similar.

Based on the assumption that head motion is small, some arrangements are such that they adopt a widely used quadratic polynomial model for E(x,y) and a linear model for H(m,n).

According to some implementations, a progressive gaze estimation process can be expressed as:

$${W ( {t}^{1} , {t}^{2} , … {t}^{M} )}^{T} = W T = {W ( f ( {e}^{1} ) , f ( {e}^{2} ) , … f ( {e}^{M} ) )}^{T} = WVc \qquad [ 2 ]$$

where
W is the diagonal weight matrix.
t^{I}, I=1, 2 . . . M is the target position corresponding to the eye feature e^{I} and I is the target index.
T∈R^{M×2} is the matrix of target coordinates from M gaze fixation samples (e^{I}, t^{I}).
V∈R^{M×N} is an observation matrix whose rows are constructed from the quadratic form of relative pupil coordinate (x^{I},y^{I}) and linear form of facial anchor coordinate (m^{I},n^{I}).c∈R^{N×2} is a matrix of model coefficients to be solved (2N unknowns).

For M>N the model can be solved as:

$$c = {( {V}^{T} {W}^{T} WV )}^{- 1} {V}^{T} {W}^{T} WT \qquad [ 3 ]$$

When a new gaze fixation sample (e^{M+1}, t^{M+1}) is observed, the row of the matrix T, V and W can be expanded and the model coefficient c can therefore be updated.

### Initial Model and Data Processing

To achieve instant gaze interaction, some arrangements utilise a pre-built model between eye features and gaze position.

A set of native gaze sample (NGS) data can be provided. That data may, for example, comprise data having the following format:

$$( {px}_{i}^{l} , {py}_{i}^{l} , {m}_{i}^{l} , {n}_{i}^{l} , {tx}_{i}^{l} , {ty}_{i}^{l} , {gx}_{i}^{l} , {gy}_{i}^{l} ) )$$

where l is the subject index, i is the sample index for each subject,

$${px}_{i}^{l} , {py}_{i}^{l}$$

comprises an eye pupil coordinate,

$${m}_{i}^{l} {n}_{i}^{l}$$

comprises a facial anchor coordinate

$${tx}_{i}^{l} , {ty}_{i}^{l}$$

comprises an associated target coordinate and

$${gx}_{i}^{l} , {gy}_{i}^{l}$$

comprises a predicted gaze coordinate.

NGS data from a total number of X subjects is provided (as set out in relation to FIG. 1). The NGS data can, in some arrangements, be collected from users who are cooperative and who successfully use gaze to control any interactive systems or from explicit calibration procedures.

Details about the number of samples required to support accurate model creation can be found in the experiment section below.

A first sample (NGS) for each user can, according to some arrangements, be collected when a user looks at the same position of a target on the screen. In some arrangements, this known position may comprise the centre of the screen or display, but it can be any position on the screen or display.

The first sample for each user is referred to as the initial pose sample. Before solving the initial model using equation [3] above, eye feature vectors are constructed for each subject based on their initial pose (as set out in the next section). Once such eye feature vector construction is performed, the model can be solved and a user can start to use gaze control within a system.

When the user starts to use the system, they need to trigger, by looking at, a target in, according to some arrangements, the middle of the screen or display, thus generating the initial gaze sample which can be used to build a local space coordinate system for further gaze estimation.

Details about how the local space based feature construction works can be found in the following section.

### Local Space Based Feature Construction

According to some arrangements, a gaze estimation model is provided based on a 2D eye feature mapping. Most existing methods construct eye features based on pupil-corner, and pupil-glint vectors. However, such feature vectors are relative vectors which cannot be used to reflect or determine head pose influence. To solve this problem, information comprising eye corners and, for example, facial information, such as inner corners, which can capture head motion, can be combined. However, such features are typically represented in global coordinates, which may not be captured in all gaze estimation systems. Furthermore, such representation is vulnerable to eye feature variation (for example, eye size variation and/or eye shape variation) and initial pose variation. If a user's eye feature information together with initial pose information are not included in an initial gaze model, there will be a large gaze estimation error at the start of use of a system.

To solve this problem, some arrangements may implement a feature construction method based on “local space”. Approaches aim to reduce influence of eye feature variation and initial head pose variation and improve gaze estimation accuracy at initialisation of a system.

As is illustrated in FIG. 1, for the lth subject an ith gaze sample results in collection of data comprising:

$$( {px}_{i}^{l} , {py}_{i}^{l} , {m}_{i}^{l} , {n}_{i}^{l} , {tx}_{i}^{l} , {ty}_{i}^{l} , {gx}_{i}^{l} , {gy}_{i}^{l} ) .$$

From that collected data, it is possible to construct an eye feature vector

$${e}_{i}^{l} = ( {dx}_{i}^{l} , {dy}_{i}^{l} , {dm}_{i}^{l} , {dn}_{i}^{l} )$$

using:
pupil-facial anchor vector displacement:

$${dx}_{i}^{l} = ( {px}_{i}^{l} - {px}_{o}^{l} ) - ( {m}_{i}^{l} - {m}_{o}^{l} ) , {dy}_{i}^{l} = ( {py}_{i}^{l} - {py}_{o}^{l} ) - ( {n}_{i}^{l} - {n}_{o}^{l} ) ,$$

and facial anchor displacement

$$d {m}_{i}^{l} = {m}_{i}^{l} - {m}_{o}^{l} , {dn}_{i}^{l} = {n}_{i}^{l} - {n}_{o}^{l} ,$$

from an initial pose where

$${px}_{o}^{l} , {py}_{o}^{l} , {m}_{o}^{l} , {n}_{o}^{l}$$

are the pupil and facial actor coordinate from an initial pose sample.

When a user starts to use a system, arrangements may be such that they are required to trigger a target in the middle of the screen to generate the initial gaze sample

$$( {{px}_{o}^{l}}_{,} {py}_{o}^{l} , {m}_{o}^{l} , {n}_{o}^{l} , {tx}_{o}^{l} , {ty}_{o}^{l} , {gx}_{o}^{l} , {gy}_{o}^{l} )$$

which can be used to build a local coordinate system based on

$$( {px}_{o}^{l} , {py}_{o}^{l} ) and ( {m}_{o}^{l} , {n}_{o}^{l} ) .$$

After an initial pose is known, a progressive gaze estimation workflow as shown in FIG. 1 may be implemented, and arrangements are such that the gaze estimation operates to map eye feature displacement from that initial pose to a screen coordinate.

The displacement mapping is a key difference between approaches and arrangements described herein and mappings based upon global coordinate representation. The local displacement mapping approach described in relation to arrangements is less affected by variation in eye features such as eye size, shape and position and head poses than global coordinate representation.

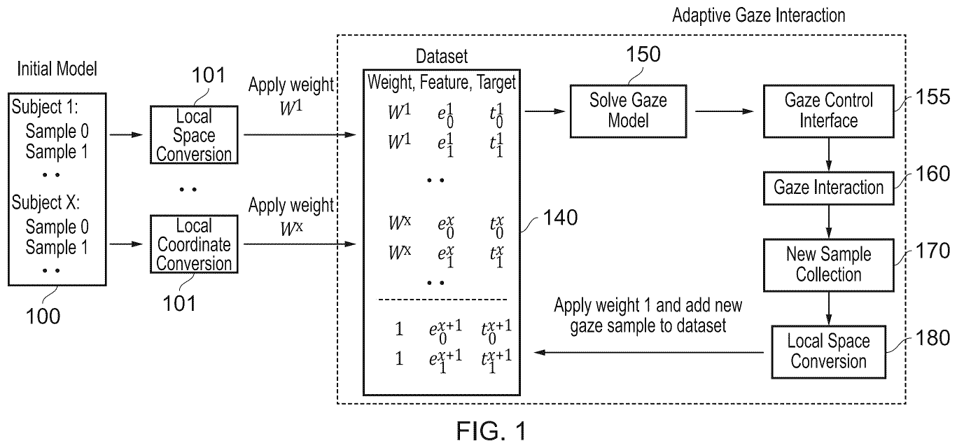

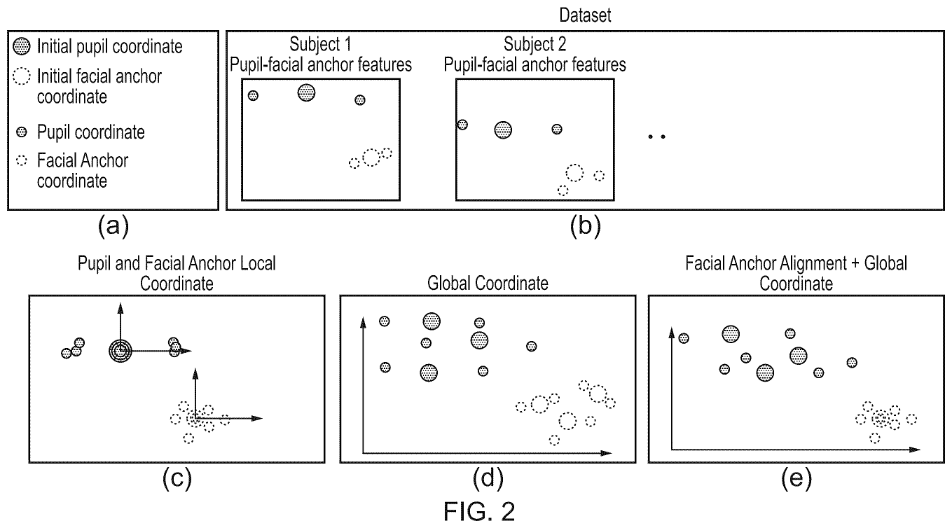

FIG. 2A to FIG. 2E provides a visual comparison of local coordinate and global coordinate based eye feature representation.

FIG. 2A provides a graphic definition of eye feature position/location data relating to an initial position of eye pupil and facial anchor. Such information forms part of a first eye image sample in relation to each user and can be mapped to, or associated with, a known target location. The first eye image samples are used, in some arrangements, to provide an “origin” of a local coordinate system for each user. As can be seen in FIG. 2A, the image data may comprise: eye pupil and facial anchor positions obtained via an initial gaze interaction.

FIG. 2B provides an illustration of eye feature distributions (in global space) in the original dataset.

FIGS. 2C, 2D and 2E show eye feature distribution in relation to all gaze samples in a dataset in local space, global space and facial anchor aligned global space respectively. The green coloured eye features represents a “test” eye feature dataset which was not included in an original dataset used to create a gaze estimation model.

FIG. 2 allows comparison of gaze sample distribution differences between local space and global space methods. FIG. 2C and FIG. 2D show gaze sample distribution differences in relation to the local and global based methods respectively.

When a user's eye feature and initial pose are not covered by an initial gaze model, as shown illustratively as the green coloured eye features in FIG. 2D, in a global coordinate system there is a large gaze error. However, if using a local space based method, no matter how different the user's initial head pose and eye features are from existing samples in the dataset, the gaze prediction will always be (gx=0,gy=0) at the outset of use of the system because the eye feature vector

$${e}_{i}^{l} = ( {dx}_{i}^{l} , {dy}_{i}^{l} , {dm}_{i}^{l} , {dn}_{i}^{l} )$$

will always be (0,0,0,0).

Once an initial image sample is collected, the sparse distribution of gaze samples in the global coordinate based dataset can still cause large gaze estimation errors, at least until sufficient samples are collected (see FIG. 2D).

Using a local space representation, samples in a dataset are aligned based on an initial pupil and facial anchor coordinate for each user (see FIG. 2C). Although global based methods can also perform a realignment process, such as facial anchor based alignment, to reduce the influence of head pose, such alignment is still influenced by eye size and shape (see FIG. 2E).

A local space representation in accordance with arrangements can align the pupil displacements and facial anchor displacements of different users based upon their initial states. An advantage of such alignment is that eye feature mapping is more closely related to the gaze direction change according to an initial pose. By contrast, a global coordinate representation is typically more influenced by head pose and eye feature variation which may not be directly related to a gaze direction change. Detailed comparison can be found in the result section.

### Experimental Platform

To demonstrate the effectiveness of various elements of proposed arrangements, and a resulting proposed gaze estimation method, experiments were carried out based on an MR compatible VR platform with content designed for patient distraction in MRI. The architecture design of the VR system shared similarities with common VR systems and comprised an interactive familiarization stage. During interactive familiarization, a user becomes familiar with basic gaze interaction tasks and learns how to navigate the system. In a virtual “lobby” an experimental system was provided comprising a virtual space with interactive content (for example, games, movies) where a user can navigate within the virtual space using gaze interaction to select content in which they are interested.

In terms of VR content, 23 gaze interaction tasks (game event trigger) were embedded into a short interactive game which guided a user from a noisy worksite (to simulate MRI scanning environment) into the virtual lobby. In the lobby, the system was configured to provide a user with a virtual cinema and a game.

In the game section, the experimental arrangement provided two games. One was a tower defence game in which a user may use their gaze to shoot enemies and collect rewards. The other game was a memory card flip game in which the user may use their gaze to flip cards and pair them.

In the movie section, several integrated cartoon movies (10 in total) were provided and a channel was built to allow a user to trigger external video resources such as YouTube, Netflix and similar.

In terms of gaze target design, approaches in accordance with some arrangements are described in more detail below.

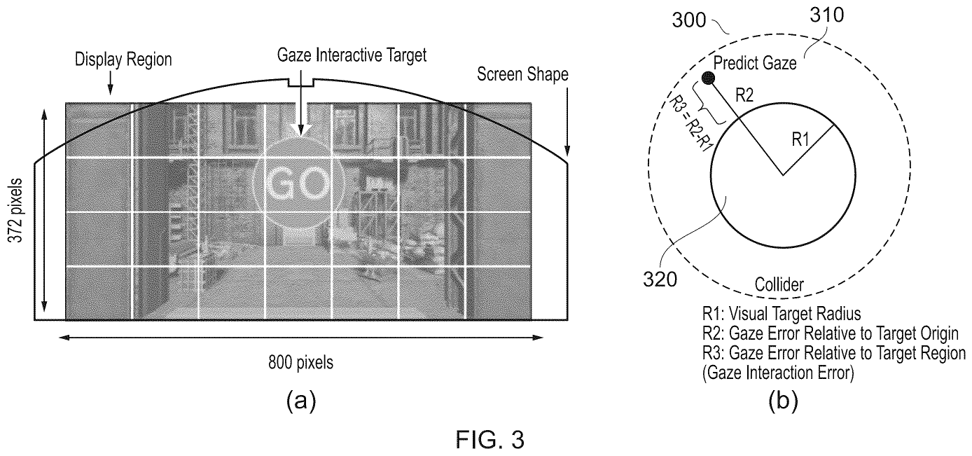

Arrangements provide a mechanism for interactive gaze target modelling, some features of which are described in more detail in relation to FIG. 3.

According to some arrangements, gaze interaction of a user with a target is based upon dwell time. When a user gaze is calculated to overlap with a “bounding collider” of a target, approaches in accordance with arrangements are such that the visual object forming the target will shrink. According to arrangements, the bounding collider size may not change during the shrinking of the visual target. According to an implementation of some arrangements, when or if the user gaze dwell time exceeds a pre-defined threshold (for example, 1.8 seconds), an event linked to the target can be triggered.

In accordance with some arrangements, during a period that a user had gaze which caused a target to shrink, it is possible to continue to collect each frame's fixation information (in other words, eye feature positions (xᵢ, yᵢ, mᵢ, nᵢ) and corresponding target positions (tx, ty) where i is the index of the corresponding frame number. To infer a moment of strongest gaze-interaction correlation from a collected eye feature sequence, according to some approaches the median of the second half of the eye feature sequence is taken as the moment of the strongest correlation between eye feature (x,y,m,n) corresponding to the target (tx, ty). The combination of the median eye feature vector and the corresponding target position is referred to as (x-m, y-n, m, n, tx, ty) which will be used as a native sample on which to build or enhance a user gaze regression model. The gaze error can be defined as distance from the predicted gaze to the visual object's boundary rather than centre because the visual targets may, for example, occupy 2-3.5 degree field of view which is about 7% to 10% of the screen width (more analysis can be found in the result section.).

FIG. 3A illustrates a screen display region in accordance with an arrangement; and FIG. 3B illustrates a gaze interaction target in accordance with an arrangement.

FIG. 3A shows a 800×372 pixel display region which, in the experimental arrangements described in more detail herein, is projected onto a 215.6 mm×64.01 mm screen. The display region in the arrangement shown is divided into a 7×4 grid. In the example shown in FIG. 3A, a gaze interactive target is placed in the centre of the grid. In this example, the gaze interactive target comprises a circular “go” button, and the centre of the ‘Go’ icon is placed at the 2nd row and the 4th column of the grid.

According to one arrangement, the interactive gaze target (shown as a large round “go” button in FIG. 3A), comprises components shown conceptually in more detail in FIG. 3B. In particular, the target 300 conceptually comprises a bounding collider 310 and a visual object region 320. The bounding collider 310 can be any shape such as box, irregular shape, oval, circle or similar and is used to capture or determine user gaze overlap with the target 300. The visual object region 320 is what a user actually sees on a display or screen (that visual object may, for example, comprise a 2D image or 3D object). A gaze prediction error (R3) is, according to some arrangements, defined as a calculated gaze position distance relative to the visual object region 320.

### Experiment Design

Two particular kinds of experiments were implemented. The first experiment type collected gaze interaction samples and provided an indication of the influence of calibration procedure on a gaze control task completion level. The second experiment type seeks to provide an indication of the effectiveness of arrangements described in achieving substantially “instant” gaze control.

According to an experiment in accordance with the first type, a user's task was to use gaze interaction to finish a familiarization procedure and explore a lobby and content (game and cinema) according to their preferences. The users were free to use the system as long as they wanted (normally 10-15 minutes).

In total 23 subjects or users were part of the test group. The 23 subjects consisted of: 5 children (age from 8 to 11); 11 adult males (age from 22 to 65) and 7 adult females (age from 22 to 50). None of the subjects had prior experience of the system.

A first group (consisting of the 5 children aged from 8-13 and 4 adults) followed an explicit calibration procedure before the familiarization. The widely used “9 point calibration” method was used. Each point of the calibration process was displayed for 3 seconds and the gap between each calibration point being shown was 2 seconds.

A second group (consisting of 14 adults) started use of a system directly from an interactive familiarization part, having performed no prior explicit calibration.

In the first group, an initial gaze model was built based on the calibration points. In the second group, an initial gaze model was built based on gaze interaction samples collected from the first group.

An experiment of the second type was also performed which focused on gaze interaction performance analysis based on gaze interaction samples collected from the 24 subjects in the first experiment (both groups). Those samples were obtained during the strongest gaze interaction moment, and therefore can be a reflection of users' attention.

To assess the effectiveness of an adaptive approach in accordance with arrangements in achieving instant gaze control and robustness, a comparison is made between:
global space based (GC);
global space and eye corner alignment based (GC+CA); and
local space based (Ours)
eye feature representation.

The experiment split the 24 subjects into two groups: a training group and a test group.

The training group is used for building an initial dataset. The test group was used to test gaze interaction performance. The training and test group split was based on subject's motion range, age and gender.

The subjects were divided into children, male and female groups. For each group, the subject's head motion range was ranked according to a displacement from their initial head pose.

The training group consisted of 8 subjects (providing a total of 358 samples). The 8 subjects were: 3 males (2 large motion and 1 small motion), 3 females (1 large motion and 2 small motion) and 2 children (1 large motion and 1 small motion).

The test group (providing a total of 675 samples) consisted of the remaining 15 subjects (3 children, 8 adult males and 4 females).

### Results

For the first experiment, in the first group, it was observed that 8 out of 12 subjects failed the test at least once (all children failed), and the person with the most failures failed 4 times. Through communication with subjects who failed the experiment, it was found that there were two main reasons for failure. The first was that the subject didn't understand the purpose of the calibration so that they didn't follow the procedure and fixate at the calibration targets. The second was that some subjects missed some calibration points for varying reasons including: that the calibration process felt boring; absence of mind; being distracted by speaking to others; being curious or anxious about the surrounding environment and similar.

To complete the test, the subjects who failed the calibration were guided through the process by explicitly asking them to follow the procedure and focus on the calibration points during the calibration procedure. Once the subjects understood the purpose of the calibration procedure, each subject was able to successfully control the system and finish the experiment without any further guidance.

In the second group, all subjects successfully achieve gaze control without explicit calibration. They all passed the familiarization stage and achieved content selection, game play and movie watching.

The experiment showed that a primary factor hindering subject's use of gaze for control in the test VR system was the calibration procedure.

None of the test subjects were found to have cognitive difficulties in understanding how to control a VR system using gaze. This included using gaze control during familiarization, VR content selection, game play and movie watching.

It will be understood that if calibration quality is good, a user will be able to immediately take control and immerse themselves in an interactive VR world using gaze control.

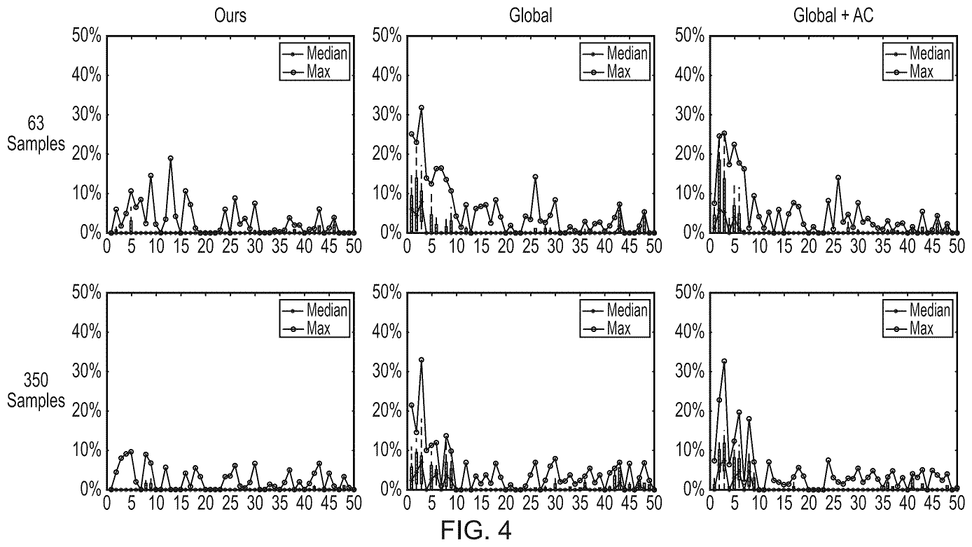

FIG. 4 is a graphical illustration of a performance comparison between various possible gaze control methods.

In particular, FIG. 4 is a graphical illustration of a performance comparison (interquartile range gaze error distribution for the test group) between GC, GC+CA and Ours (see above for definition) in relation to an initial dataset comprising a different number of samples.

The X-axis of FIG. 4 indicates an index of triggered targets. The Y-axis of FIG. 4 indicates a gaze error which is expressed as a percentage of screen width (in the experiment described, the screen width was 800 pixels).

The initial dataset for the first and second rows of FIG. 4 used 63 and 358 gaze interaction samples from the training group respectively to build an initial model. The 63 samples were evenly selected from all subjects in the training group and the corresponding target positions covered the whole region of the screen.

FIG. 4 shows that, according to the second experiment, a GC and/or GC+AC based method suffers from large gaze error in the initial phases (as can be seen in relation to the first 10 targets). The local coordinate method described herein (“ours”) appears to achieve a better result in those initial phases.

FIG. 4 also shows that the median and 75th percentile error (general performance) are around 0 throughout the whole test using “our” method, which means that predicted gazes are mostly within the target's visual region even in those initial phases.

For all methods, increasing an initial dataset's size can improve both the worst cases and the 75 percentile error. That may be expected since larger datasets include more diversity and make for more robust modelling.

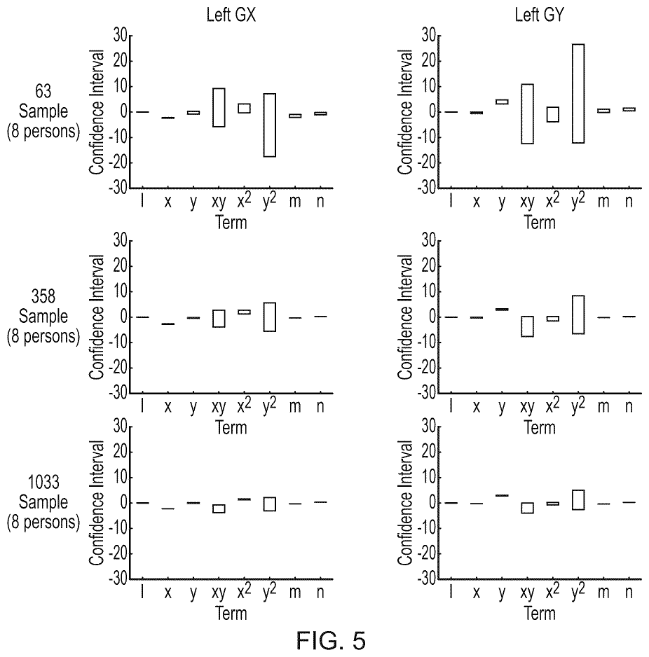

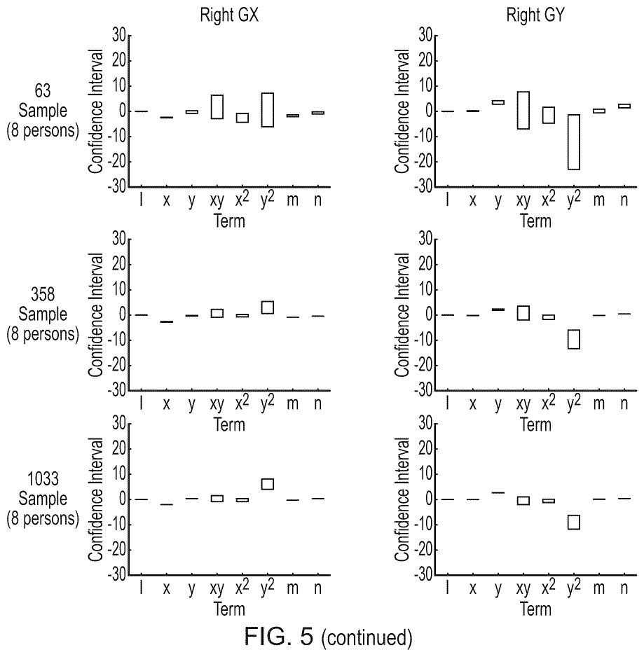

FIG. 5 illustrates graphically a confidence interval of a gaze modelling process in accordance with arrangements. In each graph of FIG. 5, the X-axis is each term's coefficient of “our” polynomial gaze model the Y-axis is the 95% confidence interval for the corresponding coefficient.

Since left gaze x, left gaze y, right gaze x and right gaze y are estimated using separate polynomials, FIG. 5 includes four columns. Each row of FIG. 5 illustrates the local gaze based model's confidence interval under different gaze sample amounts.

FIG. 5 shows how the confidence interval of a gaze model in accordance with described arrangements improves with each increase in number of samples included in an initial dataset.

Although the worst cases of a method in accordance with arrangements exceeded 10%, such worst cases are unavoidable because gaze estimation is influenced by many factors including: external distraction, absence of mind, fatigue and being uncomfortable. At least 4 subjects reported that they may have looked somewhere around the target for such reasons. 2 subjects reported that they were sensitive to the target or screen brightness in some scenes. The 75 percentile error can therefore be considered a better reflection of overall performance.

Returning now to FIG. 4, it can be seen that as the gaze interaction events increased, all three methods achieved similar results. In this instance, the models were all based on the same polynomial regression model (see equation 1) but with different coordinate systems.

Finding the best model to use in a gaze control system is a challenge. Prediction accuracy has been studied (33) (35) (36) in many models using polynomial expressions up to fourth order, and no single equation was significantly better than the rest for general usage.

As described herein, the gaze interaction performance was only assessed within one particular system. More details about traditional performance analysis of an adaptive gaze estimation process such as that described can be found in previous research (33), in which 15 subjects were asked to fixate on a sequence of targets on the screen. In that research (33), the target radius was 1.3% of screen width and prediction error was 6.20% of the screen width for 95% of all estimations.

In the experiments described herein, although the target size ranged from 7% to 10% (2-3.5 degree field of view), the gaze performance shown in FIG. 4 is consistent with the previous research (33). It can be seen, for example, that the average maximum error of “our” method is around 5%. However, the previous research relied on explicit calibration which does not support instant gaze interaction.

From a Graphic User Interface (GUI) design perspective, use of “our” method requires one initial target (in experiments described a screen centre target was used) to build the local coordinate for the current pose. As can be seen in FIG. 4, the maximum error of our method under 350 samples is around 10%. Assuming the target radius is 7%-10% (so the collider diameter should be 34%-40% to accommodate the 10% gaze error), our method can support instant gaze interaction with targets which have a 1×3 (row×column) to 1×2 layout on the screen. Based upon the 75 percentile performance of our method, the worst 75 percentile error is 3%, so the collider diameter should be 20%-26%, use of our method can support instant gaze interaction with 2×4 to 2×5 targets on the screen. More details about target size and GUI design can be found in the next section.

### User Interface Design

In this section, considerations relating to adaption of the instant gaze control framework described above are discussed. A graphical user interface (GUI) for gaze control is generally used for navigation control, for example, user interaction with a menu, and or specific buttons, and for specific tasks, for example, typing, object manipulation, game control, communication. From a GUI design perspective, methods in accordance with arrangements described above may be more suited to navigation control tasks. To adapt the instant gaze control framework in accordance with described arrangements, a GUI will be required to be configured to implement an adaptive design based on gaze error. In particular, the GUI may need to be configured such that it requires an initial single target. The initial target layout resolution may need to be sparse, for example, the results of experiments set out above indicate that approaches show support for a 1×3 target layout under max error. As gaze performance improves, a system may be configured to use a higher resolution target layout, for example, the 75 percentile gaze performance of experiments set out above supports a 2×4 to 2×5 target layout.

Although both layouts described above are sparse, they are sufficient for many navigation based tasks. For example, most VR/AR systems have an introduction or tutorial stage, in which a user needs to focus on a limited number of targets (1-2 targets) at a time in order to get familiar with the system.

From an implementation perspective, the integration an interface which utilises described approaches may require only a re-organization of a UI layout. Compared to a dynamic GUI solution, use of methods in accordance with described approaches may only require UI layout modification and there may be no need for extra logic control to adapt a GUI to gaze error.

Typically gaze interaction systems are such that when or if a large head motion happens and gaze estimation drifts, re-calibration is required. Requiring explicit recalibration is a user-unfriendly approach. It will be appreciated that approaches in accordance with described arrangements may not need implementation of an explicit re-calibration procedure. The only step needed is an extra GUI design step which comprises several buttons or triggers having a big collider (⅓-½ screen size). Based on the adaptive gaze estimation framework described above, gaze drift issues can be solved by simply interacting with the large collider associated with such buttons.

It has been found empirically that a user interacting with 1 or 2 such buttons can be sufficient to significantly improve gaze tracking performance under small head motion (details about head motion tolerance can be found in the provocation test in (33)).

Although such an extra GUI design may need to be supported by a modification of system logic, the method represents an efficient and user friendly way to maintain gaze performance compared to an explicit re-calibration strategy.

The adaptive gaze estimation approaches described herein relies on gaze interaction information to infer gaze and update the gaze model. Ideally, a user would focus on the centre of each target but often this is not possible during actual gaze interaction. There are many factors influencing gaze distribution such as: GUI element size (e.g. button, trigger) and the visual stimuli distraction (e.g. the subjects do not completely focus on the target because they are distracted by other visual stimuli). Large targets will cause a larger spread of fixations e.g. 7-8 degree field of view and small targets will cause potential calibration or target selection failure, e.g. less than 2 degree field of view cause more re-calibrations). In many cases of eye tracking research, a gaze tracker's performance is tested using simple user tasks (such as fixating at small targets on screen) rather than gaze controlled interactive applications. Traditional fixation tasks can only reflect ideal performance under controlled environment rather than interaction performance.

In the MRI compatible VR system described herein, the targets occupy 2-3.5 degree field of view which has been found to be comfortable for most subjects based on likely screen size and user head position. To reduce the potential of large spread of fixations, experiments and implementations described are such that the target visibly shrinks when user gaze is determined to overlap with the target's collider. It has been found that shrinking time and shrinking ratio influence the user experience. A long shrinking time (>2 seconds) or large shrinking ratio (>50%) may cause a user's gaze to leave the target before it is triggered. A short shrinking time may cause mis-selection (Midas Touch problem). The optimal value found for the system described above was found to be around 1.5-1.8 seconds for shrinking time and 35% for shrinking ratio.

### SUMMARY

It will be appreciated that the methods in accordance with described approaches do not, in general, rely on any specific hardware input, provision of full facial information, long hours of offline training, or irrelevant extra user tasks. The main limitation of the described methods are that they only work under small variations in user head motion. With the increasing use of near-eye eye tracker (VR, AR, MR etc.), the application of small head motion based gaze estimation techniques are of more relevance. Approaches described above can significantly contribute to this emerging field and highlight the importance of instant gaze interaction in gaze control interface research.

It can be seen that in all approaches (explicit calibration, implicit calibration and adaptive calibration) the gaze prediction became better with increased interaction samples. However, performing an explicit calibration stage is not an ideal way of supporting efficient Human Computer Interaction.

From a user interaction perspective, a calibration free approach in accordance with described approaches can allow users to immediately engage with a system using gaze interaction. Approaches support such immediate interaction by placing interactive objects in two or three areas of the screen, each of those targets or interactive objects being such that they are, for example, associated with a large degree of gaze error initially, for example, by providing an interactive object with an associated collider which is larger than a visual part of the object. The collider size may, for example, have a half screen collider size.

When designing an interactive application, a calibration free gaze estimation process in accordance with described approaches could, for example, be easily embedded into normal VR, AR or XR user tasks. Such tasks include, for example, initial setup, introduction, familiarization, tutorial system and navigation system, all of which can be components which enable users to get familiar with control features.

It will be appreciated that target distribution and display order may influence gaze model stabilization speed. A location pattern relating to target display location(s) can influence performance of a gaze model.

It is possible to run a calibration free gaze estimation process by placing targets on a display in re-assurance order. Such a re-assurance pattern is designed to prioritise ensuring a gaze model operates well in a specific region or direction first. To secure operation in an x-direction, a target may be shown in the middle, then left and right, or right and left of a display. To secure operation in the y-direction, a target may be shown in the middle, then top and bottom, or bottom and then top. Isolating gaze movement in a single direction helps to reassure the model that it is predicting well in that direction.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

### REFERENCES

6. Sugano, Y., Matsushita, Y., Sato, Y. & Koike, H. An incremental learning method for unconstrained gaze estimation. In European conference on computer vision, 656-667 (Springer, 2008).
7. Kasprowski, P. & Harezlak, K. Implicit calibration using predicted gaze targets. In Proceedings of the Ninth Biennial ACM Symposium on Eye Tracking Research & Applications, 245-248 (2016).
8. Huang, M. X., Kwok, T. C., Ngai, G., Chan, S. C. & Leong, H. V. Building a personalized, auto-calibrating eye tracker from user interactions. In Proceedings of the 2016 CHI Conference on Human Factors in Computing Systems, 5169-5179 (2016).
9. Sugano, Y., Matsushita, Y. & Sato, Y. Appearance-based gaze estimation using visual saliency. IEEE transactions on pattern analysis machine intelligence 35, 329-341 (2012).
10. Wang, K., Wang, S. & Ji, Q. Deep eye fixation map learning for calibration-free eye gaze tracking. In Proceedings of the ninth biennial ACM symposium on eye tracking research & applications, 47-55 (2016).
11. Hiroe, M., Yamamoto, M. & Nagamatsu, T. Implicit user calibration for gaze-tracking systems using an averaged saliency map around the optical axis of the eye. In Proceedings of the 2018 ACM Symposium on Eye Tracking Research & Applications, 1-5 (2018).
12. Shih, S.-W. & Liu, J. A novel approach to 3-d gaze tracking using stereo cameras. IEEE Transactions on Syst. Man, Cybern. Part B (Cybernetics) 34, 234-245 (2004).
13. Hansen, D. W. & Ji, Q. In the eye of the beholder: A survey of models for eyes and gaze. IEEE transactions on pattern analysis machine intelligence 32, 478-500 (2009).
14. Yoo, D. H. & Chung, M. J. A novel non-intrusive eye gaze estimation using cross-ratio under large head motion. Comput. Vis. Image Underst. 98, 25-51 (2005).
15. Hansen, D. W., Agustin, J. S. & Villanueva, A. Homography normalization for robust gaze estimation in uncalibrated setups. In Proceedings of the 2010 symposium on eye-tracking research & applications, 13-20 (2010).
16. Kang, J. J., Guestrin, E. D., Maclean, W. J. & Eizenman, M. Simplifying the cross-ratios method of point-of-gaze estimation. CMBES Proc. 30 (2007).
17. Coutinho, F. L. & Morimoto, C. H. Free head motion eye gaze tracking using a single camera and multiple light sources. In 2006 19th Brazilian Symposium on Computer Graphics and Image Processing, 171-178 (IEEE, 2006).
18. Arar, N. M., Gao, H. & Thiran, J.-P. A regression-based user calibration framework for real-time gaze estimation. IEEE Transactions on Circuits Syst, for Video Technol. 27, 2623-2638 (2016).
19. Kang, I. & Malpeli, J. G. Behavioral calibration of eye movement recording systems using moving targets. J. neuroscience methods 124, 213-218 (2003).
20. Pfeffer, K., Vidal, M., Turner, J., Bulling, A. & Gellersen, H. Pursuit calibration: Making gaze calibration less tedious and more flexible. In Proceedings of the 26th annual ACM symposium on User interface software and technology, 261-270 (2013).
21. Blignaut, P. Using smooth pursuit calibration for difficult-to-calibrate participants. J. Eye Mov. Res. 10 (2017).
22. Land, M. & Tatler, B. Looking and acting: vision and eye movements in natural behaviour (Oxford University Press,
   2009).
32. Redmon, J., Divvala, S., Girshick, R. & Farhadi, A. You only look once: Unified, real-time object detection. In Proceedings of the IEEE conference on computer vision and pattern recognition, 779-788 (2016).
33. Qian, K. et al. An eye tracking based virtual reality system for use inside magnetic resonance imaging systems. Sci. reports 11, 1-17 (2021).

1. A method of auto-calibrating an eye gaze tracking system for a user, the method comprising:
providing a model linking eye data with a gaze position on a display, wherein the model links user eye feature locations and user head locations to gaze locations;
acquiring user eye data and transforming the acquired user eye data into a local eye image space to obtain an indication of user eye feature location and an indication of user head location in a centralised eye image space;
providing an icon at a known position on the display, the icon being associated with a collider area;
transforming the known position to accord with the local eye image space;
estimating, based upon the model and the transformed acquired user eye data and the transformed known position, whether a user gaze position falls within the collider area;
based on determining the estimated user gaze position falls within the collider area, associating the transformed acquired user eye data with the transformed known position; and
adapting the model based upon the transformed known position and associated transformed acquired eye data.

2. A The method according to claim 1, wherein the indication of user eye feature location comprises an indication of user eye pupil location.

3. The method according to claim 1, wherein the indication of user head location comprises an indication of a location of a facial anchor.

4. The method according to claim 1, wherein the model comprises a model which maps the local eye image space to a display gaze position which has been transformed into an equivalent transformed gaze space.

5. The method according to claim 1, wherein the local eye image space comprises a coordinate system in which an eye tracking origin coordinate used when acquiring user data from an eye image is transformed and scaled to map to an image centre.

6. The method according to claim 1, wherein transformed known positions comprise display locations which have been transformed to account for origin and scale transformations applied to data from an eye image.

7. The method according to claim 1, wherein the icon comprises a visual target and an associated virtual collider area.

8. The method according to claim 7, wherein the associated virtual collider area has a diameter at least twice the diameter of the visual target.

9. The method according to claim 1, wherein the collider area is centred upon the known position.

10. The method according to claim 1, wherein the icon comprises an adaptive icon at the known position on the display, the adaptive icon being configured to adapt when the estimated user gaze position falls within the collider area.

11. The method according to claim 10, wherein the method comprises: adapting the icon while the estimated user gaze position is determined to fall within the collider area.

12. The method according to claim 1, wherein the method further comprises performing steps comprising:
acquiring further user eye data and transforming the further acquired user eye data into the local eye image space to obtain another indication of user eye feature location and another indication of user head location in the centralised eye image space;
providing an additional icon at a further known position on the display, the additional icon being associated with an additional collider area and transforming the further known position to accord with the local eye image space;
estimating, based upon the model and transformed further acquired user eye data, and the transformed further known position whether an additional user gaze position falls within the additional collider area;
based on determining the additional estimated user gaze position falls within the additional collider area, associating the further transformed acquired further user eye data with the transformed further known position; and
further adapting the model based upon the transformed further known position and associated transformed further acquired eye data.

13. The method according to claim 12, wherein the method comprises iterating the model linking eye data with a gaze positions on the display to auto-calibrate for a user.

14. The method according to claim 13, wherein the method comprises changing a size of the collider area as part of a series of iterative steps to refine the auto-calibration of the eye gaze tracking system for the user.

15. The method according to claim 14, wherein changing the size of the collider area comprises: reducing a relative size of the collider area compared to the icon.

16. The method according to claim 12, wherein the known position comprises a position substantially in a centre of the display.

17. The method according to claim 16, wherein the further known position comprises a position at a periphery of the display.

18. (canceled)

19. An auto-calibrated gaze estimation apparatus comprising at least one processor; and at least one memory storing instructions that, when executed by the at least one processor, cause the gaze estimation apparatus to:
provide a model linking eye data with a gaze position on a display, wherein the model links user eye feature locations and user head locations to gaze locations;
acquire user eye data and transforming the acquired user eye data into a local eye image space to obtain an indication of user eye feature location and an indication of user head location in a centralised eye image space;
provide an icon at a known position on the display, the icon being associated with a collider area;
transforming the known position to accord with the local eye image space;
estimate, based upon the model and the transformed acquired user eye data and the transformed known position, whether a user gaze position falls within the collider area;
based on determining the estimated user gaze position falls within the collider area, associate the transformed acquired user eye data with the transformed known position; and
adapt the model based upon the transformed known position and associated transformed acquired eye data.
